(19) United States
(12) Patent Application Publication
(10) Pub. No.: US 2003/0087297 A1
(43) Pub. Date: May 8, 2003

(54) Biochip and genetic sequence measuring equipment using the biochip

(75) Inventors: Minoru Ootsubo, Tokyo (JP); Takeo Tanaami, Tokyo (JP)

Correspondence Address: ARMSTRONG,WESTERMAN & HATTORI, LLP, 1725 K STREET, NW, SUITE 1000, WASHINGTON, DC, 20006, US

(73) Assignee: YOKOGAWA ELECTRIC CORPORATION, Tokyo (JP)

(21) Appl. No.: 10/286,817

(22) Filed: Nov. 4, 2002

(30) Foreign Application Priority Data
Nov. 8, 2001 (JP) 2001-343020

(51) Int. Cl. C12Q 1/68; C12M 1/34

(52) U.S. Cl. 435/6; 435/287.2; 205/777.5

(57) ABSTRACT

The present invention is characterized by that a biochip in which a plurality of biopolymers is arranged, has a transparent layer having a fluorescence enhancing function on a metal layer which is also used as a one-side electrode for implementing hybridization.

### BACKGROUND OF THE INVENTION

1. Field of the Invention

The present invention relates to a biochip for examining the sequence of genes of biopolymers such as DNA and proteins, and to genetic sequence measuring equipment using the biochip.

2. Description of the Prior Art

Through hybridization by passing unknown DNA over a substrate on which known DNA is fixed, the unknown DNA can be bound to a corresponding DNA sequence. In this case, the unknown DNA sequence bound to the known DNA can be known by binding a fluorescent reagent to the unknown DNA.

As shown in FIG. 1(a), if a positive voltage is applied to electrode 1 on which known DNA 2 is attached, unknown DNA 3 is attracted to the side of electrode 1 as shown in FIG. 1(b) because DNA is negatively charged. This makes hybridization, which previously took a few hours to be completed, possible in tens of seconds.

As equipment that can make hybridization speed higher by applying this principle, for example, there is the measuring equipment that measures genetic sequences mentioned in Japanese Patent Application Laid Open No. 2002-85095 proposed by the applicant for the application concerned. This measuring equipment is configured as shown in FIG. 2. The inside of cartridge 11 formed with an insulator is leak proof and filled with a liquid in which known DNA 2 and unknown DNA 3 are mixed.

Known DNA 2 is fixed to the wall surface of cartridge 11 as shown in FIG. 2(a). When a voltage is applied from voltage source 14 between positive electrode 12 and negative electrode 13 positioned on either side of cartridge 11, the suspended unknown DNA 3 which it contains, since being negatively charged, is attracted by and comes close to positive electrode 12 as shown in FIG. 2(b). In such a manner, the speed of hybridization can be made higher.

Also, if unknown DNA 3 is labeled with a fluorescent material in advance and the exciting light is irradiated onto the DNA 3 to emit fluorescence, the more intense the detected fluorescence, the higher the detecting sensitivity of that system. Notably, the quantification of smaller traces of proteins and nucleic acids becomes possible. For this reason, enhancing the intensity of fluorescence from the fluorescent material whose quantity is equal to that of the fluorescent material before enhancement is very significant.

In the U.S. Pat. No. 4,649,280, a fluorescence enhanced chip is described, in which the intensity of fluorescence generated from a fluorescent material 24 can be enhanced by adopting a structure in which layers of metal 22, dielectric material 23 and fluorescent material 24 are stacked in this order on a glass substrate 21 as shown in FIG. 3.

However, there are the following problems with these conventional chips:

In chips for high speed hybridization;

(a) Since thickness of some extent is necessary for the cartridge, the distance between the electrodes becomes long thereby decreasing the intensity of the electric field.

(b) Since this configuration requires components such as a cartridge, electrodes, and others, increase of the number of components is significant.

(c) Although hybridization speed is increased, sensitivity is not necessarily improved.

On the other hand, in fluorescence enhanced chips, although sensitivity is improved, hybridization speed is not necessarily made higher.

### SUMMARY OF THE INVENTION

The purpose of the present invention is to realize biochips and genetic sequence measuring equipment in which hybridization of higher speed and higher sensitivity can be achieved by implementing hybridization employing a specific fluorescent enhancement part and using the metal layer of the fluorescent enhancement part also as an electrode for solving the above mentioned problems.

### BRIEF DESCRIPTION OF THE DRAWINGS

FIG. 1 shows a drawing illustrating the attraction of DNA towards the electrode.

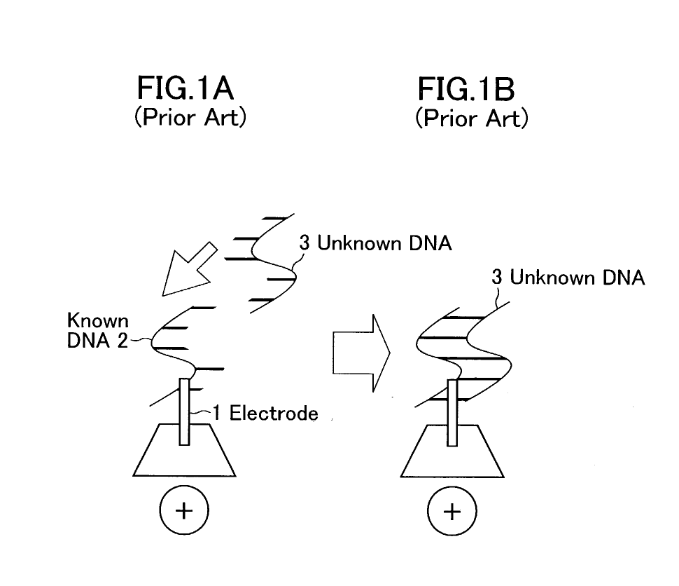

FIG. 2 is a configuration drawing showing an example of conventional measuring equipment.

FIG. 3 is a configuration drawing showing an example of conventional fluorescent enhancement chips.

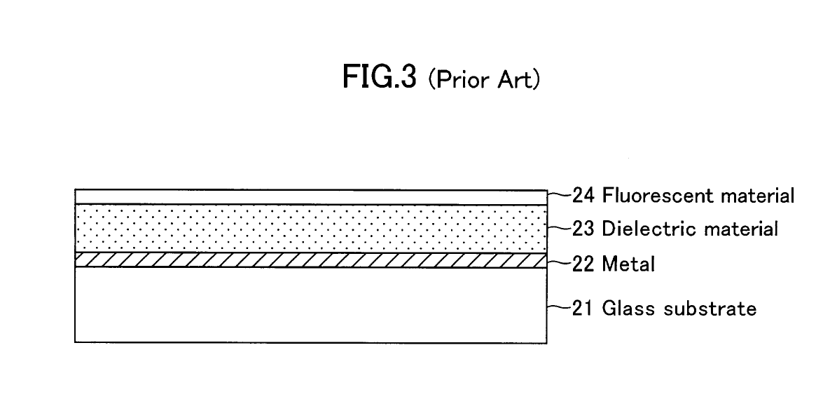

FIG. 4 is a drawing showing the essential part of measuring equipment using a biochip indicating an embodiment of the present invention.

FIG. 5 is a drawing showing a sectional enlargement of the fluorescent enhancement part.

FIG. 6 is a drawing showing the essential part of measuring equipment using a biochip indicating another embodiment of the present invention.

### DETAILED DESCRIPTION OF THE PREFERRED EMBODIMENTS

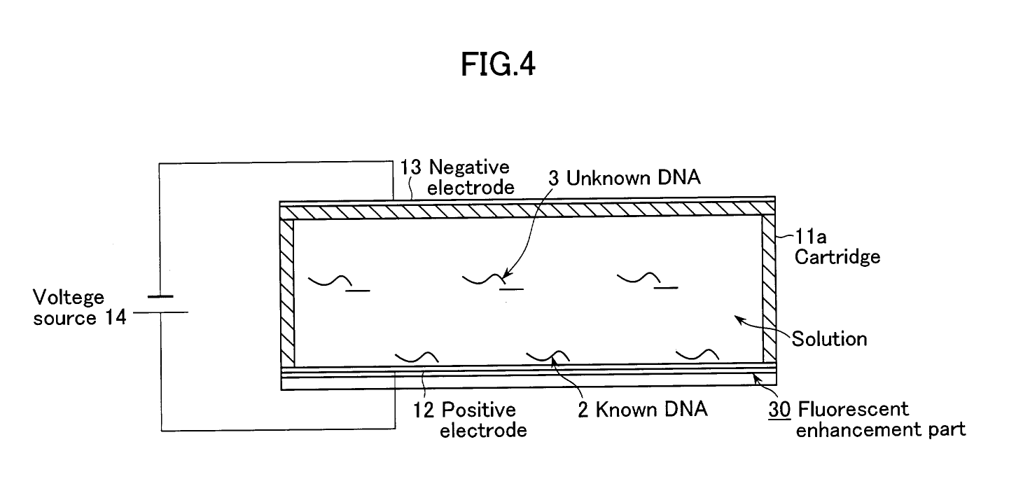

The present invention will be described below in detail using drawings. FIG. 4 is a drawing showing the essential part of measuring equipment using a biochip indicating an embodiment of the present invention.

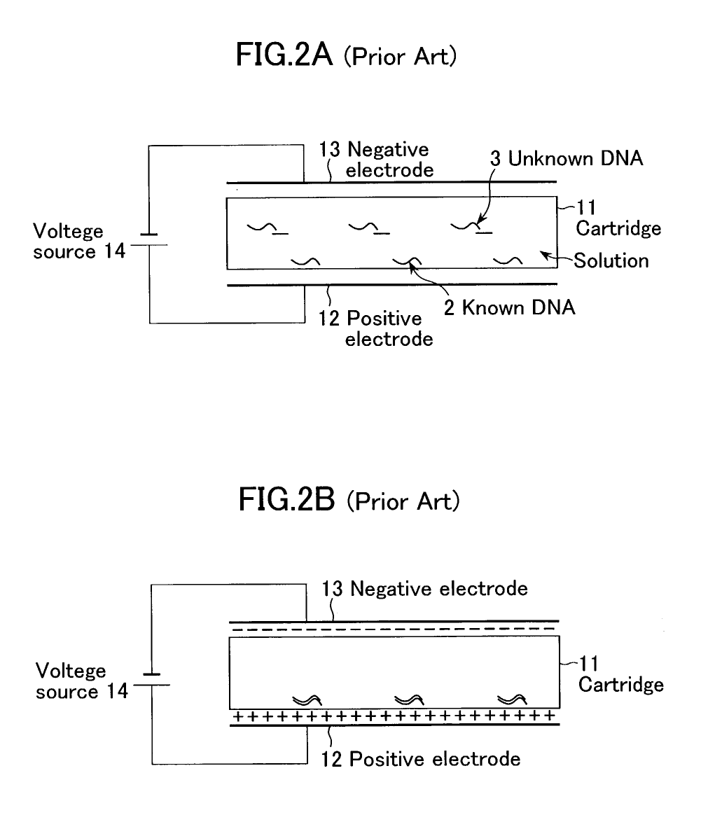

In FIG. 4, elements identical to those of FIG. 2 are referenced alike. Elements different from those in FIG. 2 are of such construction that the bottom of cartridge 11a formed with transparent materials comprises the fluorescent enhancement part 30, and negative electrode 13 is constructed in a detachable manner and is mounted on the upper surface of cartridge 11a.

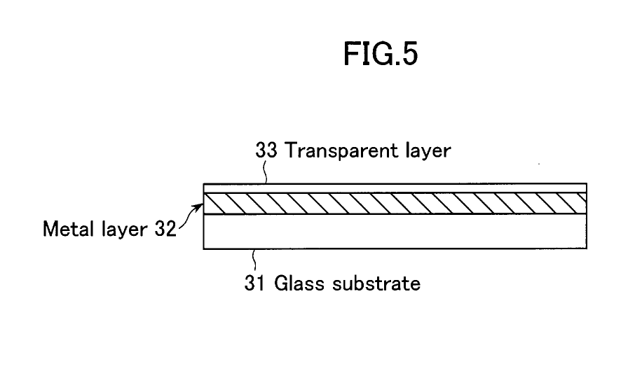

FIG. 5 is a drawing showing a sectional enlargement of the fluorescent enhancement part 30. This fluorescent enhancement part 30 has a structure, in which metal layer 32 and transparent layer 33 are stacked on glass substrate 31, and is mounted on the surface of the bottom of cartridge 11a in a leak proof manner with transparent layer 33 situated on the inner side.

In this case, metal layer 32 has the effect of reflecting mirror actions for fluorescence enhancement and is also used as the positive electrode for hybridization. In addition, transparent layer 33 also serves as the insulator in hybridization.

In this case, if transparent layer 33 has a prescribe thickness, for example, ¼ of the wavelength of the fluorescence or a thickness obtained by adding an integer multiple of ½ of the wavelength to the above ¼ of the wavelength [that is, a thickness of ¼+i/2 (where i=0, 1, 2, . . . ) of the fluorescence wavelength], the transparent layer has the function of enhancing the fluorescence intensity. This transparent layer is made of materials such as glass, gel or resin. Metal layer 32 is made of silver (Ag), aluminum (Al) or the like.

Actions in the configuration shown in FIG. 5 will be described below. Known DNA 2 is fixed to the surface of transparent layer 33 of fluorescent enhancement part 30. Metal layer 32, which is provided for enhancing fluorescence intensity and insulated from the solution, is utilized as the positive electrode. This positive electrode is counter to negative electrode 13 and thus configuration is such that there is a solution containing biopolymers such as charged DNA in the region between these electrodes.

An electric field is developed by applying a voltage across the above electrodes from voltage source 14. Since DNA is negatively charged, it is attracted toward the positive electrode and thus unknown DNA 3 is hybridized with known DNA being in relation to the unknown DNA in a complementary manner.

After hybridization, voltage application to the electrodes is stopped and negative electrode 13 is removed from cartridge 11a.

Since unknown DNA 3 bound to known DNA is labeled with fluorescent material, that unknown DNA sequence can be measured by carrying out fluorescence measurement of fluorescent enhancement part 30 of cartridge 11a.

The present invention is not to be restricted to the above embodiments but may be subject to more changes or modifications without departing from the true spirit thereof.

For example, by employing a transparent electrode as negative electrode 13, DNA sequence measurement after hybridization can be carried out without removing the electrode.

Further, as metal layer 32, silver or aluminum can be used.

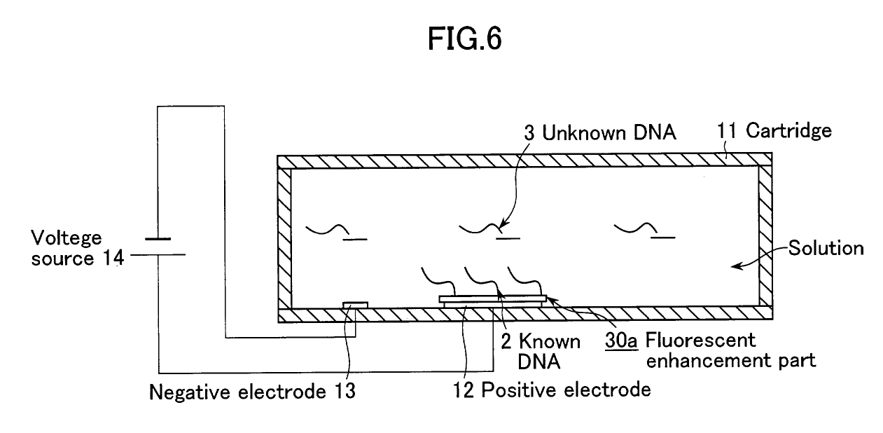

In addition, although the above embodiments employ the so called electric field accelerating type method that increases hybridization speed by applying an electric field to a solution, the current accelerating type method as shown in FIG. 6 can also be employed. In FIG. 6, number 13a shows a negative electrode and number 30a shows a fluorescent enhancement part composed of metal layer 32 and transparent layer 33. Negative electrode 13a and metal layer 32 (also used as the positive electrode) are mounted to the inner wall surface of cartridge 11 which is made of insulating material. In addition, negative electrode 13a can be mounted anywhere on the inner surface of the cartridge as long as it is positioned separate from metal layer 32.

In such a configuration, if known DNA 2 is fixed on the surface of transparent layer 33 of fluorescent enhancement part 30 similar to the case in FIG. 4 and a voltage is applied from voltage source 14 (although current flows in the solution in this case), the negatively charged unknown DNA 3 is attracted toward the positive electrode (metal layer 32) and hybridized with known DNA 2 which is related to DNA 3 in a complementary manner.

Further, the structure of transparent layer 33 shown in FIG. 4 and FIG. 6 is not limited to glass and gel or resin can also be used. The voltage applied from voltage source 14 is not limited to a DC voltage but can also be an AC voltage or a pulse voltage.

Furthermore, known DNA may also be fixed, not on the surface of transparent layer 33, but to ground work metal layer 32. This technique is specifically effective in cases where this transparent layer is made of gel.

As described above, the present invention has the following effects:

(1) Both the electric field accelerating type and the current accelerating type of hybridization can be achieved at higher speed simultaneously with higher sensitivity by employing a fluorescent enhancement part and also using the metal layer of that fluorescent enhancement part as an electrode.

(2) Since the metal layer of the fluorescent enhancement part is also used as an electrode, it is not required to provide the positive electrode separately as in previous designs and the number of components is reduced.

(3) Because insulation is provided with a thin transparent layer, the distance between the electrodes can easily be shortened, and miniaturization of the cartridge and high speed hybridization can easily be achieved.

What is claimed is

1. A biochip in which a plurality of biopolymers is arranged, having a transparent layer that has a fluorescence enhancing function on a metal layer which is also used as one electrode for implementing hybridization.

2. A biochip in accordance with claim 1, wherein said transparent layer has a thickness equal to [¼+i/2 (i=0, 1, 2, . . . )] of said fluorescence wavelength.

3. A biochip in accordance with claim 1 or claim 2, wherein said metal layer is made of silver or aluminum and said transparent layer is made of glass, gel or resin.

4. A biochip in accordance with any of claims 1 to 3, wherein said hybridization is configured to be implemented in the electric field accelerating type or the current accelerating type of hybridization.

5. A biochip in accordance with claim 4, wherein the voltage applied to the electrodes in said electric field accelerating type of hybridization is a DC voltage, an AC voltage or a pulse voltage.

6. Genetic sequence measuring equipment configured to arrange a plurality of biopolymers and to measure the genetic sequence of the biopolymer by implementing hybridization comprising:
a cartridge filled with solution containing biopolymers,
a biochip mounted inside this cartridge and formed with a metal layer which is also used as an electrode for implementing hybridization, and a transparent layer having a fluorescence enhancing function stacked on said metal layer and on this transparent layer known biopolymers being fixed,
a negative electrode for hybridization mounted to said cartridge, and
a means for applying a voltage across said metal layer of said biochip and said negative electrode;
and binding unknown biopolymers labeled with fluorescent material and suspended in said solution with known biopolymers fixed to said transparent or metal layer in a complementary manner to enable measurement of the genetic sequence of the biopolymers.

7. Genetic sequence measuring equipment in accordance with claim 6, wherein said metal layer of said biochip is made of silver or aluminum and said transparent layer of said biochip is made of glass or gel or resin.

8. Genetic sequence measuring equipment in accordance with claim 6, wherein said transparent layer of said biochip has a thickness equal to [¼+i/2 (i=0, 1, 2, . . . )] of said fluorescence wavelength.

9. Genetic sequence measuring equipment in accordance with any of claims 6 to 8, wherein said negative electrode can be removed from said cartridge after hybridization or is formed with a transparent electrode (wherein removal is not necessary).

10. Genetic sequence measuring equipment in accordance with any of claims 6 to 9, wherein said means for applying a voltage is configured to enable the application of DC voltage or AC voltage or pulse voltage.

11. Genetic sequence measuring equipment in accordance with claim 6, wherein said negative electrode is mounted to the inner surface of said cartridge and configured so that said hybridization is implemented in the current accelerating type hybridization.
